(19) United States
(12) Patent Application Publication
(10) Pub. No.: US 2010/0023348 A1
(43) Pub. Date: Jan. 28, 2010

(54) REMOTELY TAKING REAL-TIME PROGRAMMATIC ACTIONS RESPONSIVE TO HEALTH METRICS RECEIVED FROM WORN HEALTH MONITORING DEVICES

(75) Inventors: Christopher J. Hardee, Raleigh, NC (US); Donna C. Hardee, Raleigh, NC (US); Adam Roberts, Moncure, NC (US)

Correspondence Address: PATENTS ON DEMAND, P.A. IBM-RSW, 4581 WESTON ROAD, SUITE 345, WESTON, FL, 33331, US

(73) Assignee: INTERNATIONAL BUSINESS MACHINES CORPORATION, Armonk, NY (US)

(21) Appl. No.: 12/177,594

(22) Filed: Jul. 22, 2008

(51) Int. Cl. G06Q 50/00; A61B 5/00

(52) U.S. Cl. 705/3; 600/300

(57) ABSTRACT

Health metrics can be received from at least one person wearing a health monitoring device in an unencumbered fashion that permits free motion. The health monitoring device can wirelessly convey the heath metrics to a remotely located computing device. The received health metrics can be compared against at least one configurable, yet previously established threshold. A change in a situation proximate to the person can be inferred based upon comparison results. A programmatic event can be fired based upon the inferred change. At least one programmatic action can be automatically initiated responsive to the firing of the programmatic event, wherein the programmatic action initiates a response to the inferred change.

### BACKGROUND OF THE INVENTION

The present invention relates to the field of health monitoring, more particularly to remotely taking real-time programmatic actions responsive to health metrics received from worn health monitoring devices.

There are many situations when it can be desirable to automatically (through passive actions) inform another when ones heath status changes dramatically.

For example, bodyguards, police, military personnel, security guards, and others are often employed to ensure the safety of others within a potentially hazardous environment. Multiple personnel can often coordinate actions within this environment with each other. Typically radio communications are maintained for coordination purposes. These communications, however, require active actions by each individual. Should one of these individuals become unavailable for whatever reason, others are only made aware of this lack when that individual is unreachable or fails to respond to a communication attempt. This can be problematic as it delays an awareness of a potential weakness in a security parameter, which can be situationally critical.

Additionally, often coordinating agents fail to notify others of potentially significant information, which would be available through body health metrics. For example, if a person is scared, his/her heart rate increases, yet that person may not voice their fear. In another example, if a person is feeling ill, physiological symptoms often manifest before a harmful event occurs. For example, a person standing outdoors for extended periods on a hot day can experience heat stroke, which can eventually cause that person to pass out.

Security related situations are just one of many types of situation where it can be advantageous to have one's change in health conveyed to another. For instance, a number of elderly people living alone wear an alerting device, which when pressed notifies a remote agent/company that help is needed. These devices are only useful if the user is sufficiently alert and able to activate the alerting device. A better (yet currently non-existing) solution is to provide a health monitoring device to the elderly individual that automatically broadcasts a distress signal (without user actions) when body metrics indicate a distress situation exists.

### BRIEF DESCRIPTION OF THE SEVERAL VIEWS OF THE DRAWINGS

FIG. 1 is a schematic diagram of a system for remotely monitoring changes in bodily functions and responding to emergency situations in accordance with an embodiment of the inventive arrangements disclosed herein.

FIG. 2 illustrates scenarios for remotely monitoring changes in bodily functions and responding to emergency situations in accordance with an embodiment of the inventive arrangements disclosed herein.

FIG. 3 is a flow chart of a method for remotely monitoring changes in bodily functions and responding to emergency situations in accordance with an embodiment of the inventive arrangements disclosed herein.

### DETAILED DESCRIPTION OF THE INVENTION

The present invention can allow for remotely monitoring changes in bodily functions, such as, but not limited to, heart rate, blood pressure, galvanic skin response, blood sugar level, and the like. Any number of health characteristics can be monitored by the present invention. A health monitoring device can be worn by or implanted into someone whose health should be monitored. The health monitoring device can monitor bodily changes and report them to an external device or devices. In some cases, the health monitoring device can alert other health monitoring devices within range.

For example, all of the members of a military unit can be equipped with a health monitoring device. When a significant change in monitored metrics occurs, the other members of the unit will be notified. In other cases, the health monitoring device can contact a remote computing device or server. In one embodiment, notifications can occur in real time.

The computing device or server that receives the alert can respond accordingly. For example, thresholds can be established against metrics and programmatic events can be automatically triggered when a threshold is exceeded. Further, group thresholds can be established that trigger selective programmatic actions only when more than one monitored user experiences a change in health metrics characteristic of a given situation. For example, when multiple monitored users experience symptoms characteristic of a gas leak, carbon monoxide poisoning, etc., suitable actions can be automatically triggered to counteract such a situation.

In some cases, the computing device or server can contact an emergency medical service, law enforcement, or the like. In one embodiment, when a computing device or server is contacted, the computing device or server can combine all known information regarding the reporting health monitoring devices and provide the information in a graphical user interface. Security and privacy protocols can be optionally implemented to prevent the health monitoring data from being used in ways adverse to monitored individuals.

The present invention may be embodied as a method, system, or computer program product. Accordingly, the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present invention may take the form of a computer program product on a computer-usable storage medium having computer-usable program code embodied in the medium. In a preferred embodiment, the invention is implemented in software, which includes but is not limited to firmware, resident software, microcode, etc.

Furthermore, the invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. Examples of a computer-readable medium include a semiconductor or solid state memory, magnetic tape, a removable computer diskette, a random access memory (RAM), a read-only memory (ROM), an erasable programmable read-only memory (EPROM or Flash memory, a rigid magnetic disk and an optical disk. Current examples of optical disks include compact disk—read only memory (CD-ROM), compact disk—read/write (CD-R/W) and DVD. Other computer-readable medium can include a transmission media, such as those supporting the Internet, an intranet, a personal area network (PAN), or a magnetic storage device. Transmission media can include an electrical connection having one or more wires, an optical fiber, an optical storage device, and a defined segment of the electromagnet spectrum through which digitally encoded content is wirelessly conveyed using a carrier wave.

Note that the computer-usable or computer-readable medium can even include paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Computer program code for carrying out operations of the present invention may be written in an object oriented programming language such as Java, Smalltalk, C++ or the like. However, the computer program code for carrying out operations of the present invention may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN) or a wide area network (WAN), or the connection may be made to an external computer (for example, through the Internet using an Internet Service Provider).

A data processing system suitable for storing and/or executing program code will include at least one processor coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution.

Input/output or I/O devices (including but not limited to keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

The present invention is described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

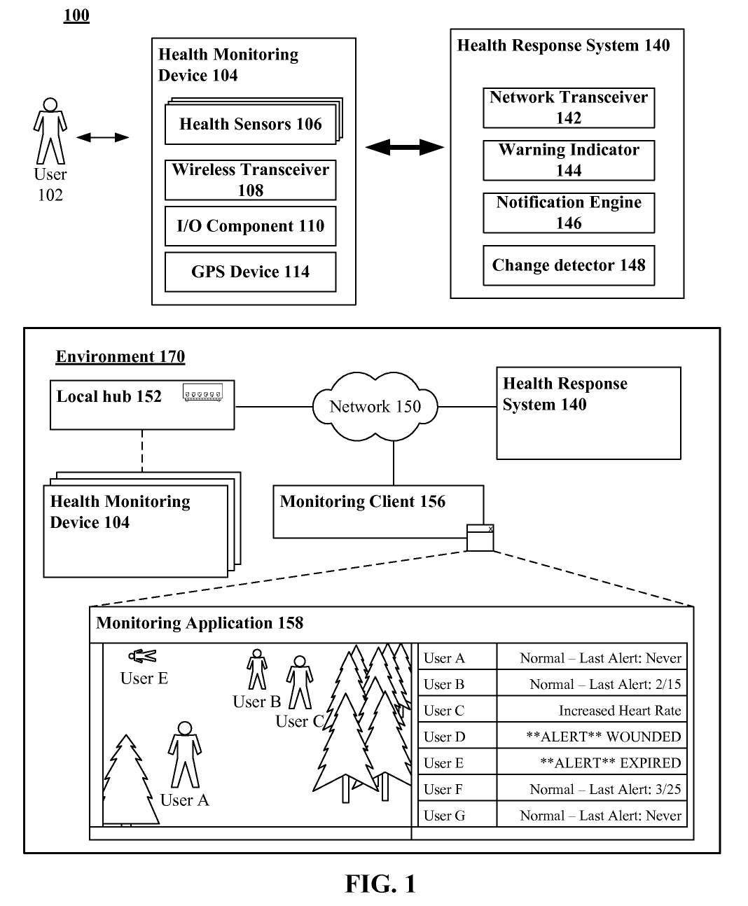

FIG. 1 is a schematic diagram of a system 100 for remotely monitoring changes in bodily functions and responding to emergency situations in accordance with an embodiment of the inventive arrangements disclosed herein. In system 100, health monitoring device 104 can be a device capable of monitoring bodily functions of user 102 and reporting the data to health response system 140. The device 104 can be unobtrusively worn in standard environments with minimal encumbrance. For example, the device 104 can be a wearable device, such as a wrist watch, as an anklet, and the like. Health response system 140 can be a remotely located system configured to receive health metrics from the device 104. These metrics can be received and processed in real-time, in near real-time, and/or after an arbitrary delay depending upon implementation specifics. The health response system 140 can be configured to perform a pre-programmed programmatic action whenever a heath metric gathered by device 104 exceeds a previously configured threshold. In one embodiment, the programmatic action can be a notification action that notifies an individual other than the user 102 of a potential situation that merits a response. In one embodiment, multiple different health monitoring devices 104 can convey health metrics to the health response system 140. Programmatic actions and/or configured thresholds of the health response system 140 can be based upon input from multiple different devices 104.

As shown, a health monitoring device 104 can include one or more health sensors 106, a wireless transceiver 108, an input/output (I/O) component 110, a global positioning system (GPS) device 114, and/or other such components. The components shown for the device 104 are not necessarily contained in a single discrete device, but can be contained within a set of different devices that are communicatively linked to each other. For example, a health sensor 106 can be embedded in a wrist watch, which communicates to a GPS 114 equipped mobile phone, which also includes the wireless transceiver, 108 and the I/O component 110. It is important that the health monitoring device 104, however configured, can be unobtrusively utilized in a deployed environment, where the user 102 can perform relatively unencumbered actions.

The health response system 140 can include a network transceiver 142, a warning indicator 144, a notification engine 146, a change detector 148, and other such components. System 140 can be implemented within a centralized hardware device, can be implemented using a set of distributed devices interoperating in a distributed computing environment, and the like. Further, components 142-148 of system 140 can vary based upon implementation specifics. For example, an optional warning indicator 144 can present a warning proximate to the geographical area of the user 102 in one situation, and can provide a silent warning not discernable from the geographic environment proximate to user 102 in another situation. The change detector 148 can compare received health metrics against any set of defined thresholds and can trigger programmatic events that are driven by comparison results.

Health sensors 106 can be responsible for monitoring the bodily functions of user 102. Health sensors 106 can include a set of sensors for doing tests on the human body to determine characteristics of bodily functions. For example, health sensors 106 can monitor galvanic skin response, heart rate, blood pressure, blood sugar levels, blood alcohol level, pupil dilation, and the like. In one embodiment, the health sensors 106 can also monitor conditions of an environment proximate to the user 102, such as ambient temperature, environmental gas composition, ambient sounds, and the like. These environmental conditions can be contextually useful when evaluating health metrics and to minimize false positive reactions.

The wireless transceiver 108 142 can be any component able to convey digitally encoded information within carrier waves to an external component. In one embodiment, the transceiver 108 can be configured to only transmit health metrics and transceiver 142 can be configured to only receive them. In another embodiment, the transceiver 108 can receive commands and/or data from remotely located device, which enable it to perform responsive actions, such as presenting an alert and/or providing messages regarding health statuses of proximate health monitored users.

Health sensors 106 can provide this data to wireless transceiver 108 for conveyance to health response system 140. Wireless transceivers 108 and 142 can be corresponding transceivers in which can communicate with each other. Wireless transceivers 108 and 142 can convert digital data into transmittable signals and can convert received signals into usable digital data. Wireless transceivers 108 and 142 can implement any form of wireless technology short or long distance. For example, wireless transceivers 108 and 142 can implement BLUETOOTH, WIFI, or the like.

Input/Output (I/O) component(s) 110 can be responsible for presenting output and receiving input to and from a user. I/O component 110 can present data to the user regarding their health. In some cases, another user can be wearing a health monitoring device in a proximate area. If the health monitoring device detects a health alert, it can notify the proximate health monitoring devices of the alert. When an alert is received, I/O component 110 can notify the user of the alert and provide associated data, such as the location, reason for the alert, and the like. I/O component 110 can include multiple components, which can provide the input/output functionality described herein. I/O component 110 can include a display and input mechanism. In one example, the display and input mechanism can be combined in the form of a touch sensitive display device. In other embodiments, the display can be a liquid crystal display (LCD) or organic light-emitting diode (OLED) display. The input mechanism can consist of a series of buttons, or controls, which can allow the user to interact with the display content. In some embodiments where health monitoring device 104 is intended for output only, the input mechanism can be omitted. I/O components 104 can also include audio transceivers (speaker and/or microphone) linked to speech processing components, such as speech recognition for audio input and speech synthesis for audio output.

GPS device 114 can be an optional component in which can make use of the Global Positioning System (GPS). Global positioning system is a collection of satellites and/or ground stations that broadcast timing and location signals from which a device 104 position can be triangulated. Other location detection technologies (e.g., LORAN) can be utilized in place of the GPS device for similar purposes.

Change detector 148 can monitor changes in health metrics received from health monitoring devices. Change detector 148 can actuate a health alert when a significant change is detected that exceeds safety thresholds. Change detector 148 can use warning indicator 144 and/or notification engine 146 to notify a user or users of the health alert. Warning indicator 144 can be a local warning indicator in the case of a health alert. Warning indicator 144 can be a visual, audible, or other kind of alarm. Warning indicator 144 can notify proximate users of a health alert. Notification engine 146 can notify external devices of a health alert. For example, notification engine 146 can notify health monitoring devices proximate to the health monitoring device in which is causing the alert. Notification engine 146 can also notify any amount of other external devices, such as one owned by an emergency medical response service.

Environment 170 represents one potential environment in which the health device 104 and health response system 140 are implemented. In environment 170, a set of health monitoring device can be communicatively linked to a local hub 152, which is connected to a network 150. Health response system 140 and a monitoring client 156 device can also be connected to network 150.

Health monitoring client 156 and related monitoring application 158 are presented to show one contemplated use of the real time health metrics. The monitoring client 156 can present a position (based upon GPS data) and heath status of a set of monitored users (Users A-G) within a geographic map. For example, a situation coordinator, military officer, etc. can utilize such a monitoring application 158 to coordinate a situation response in real-time. In one embodiment, a fly over can be presented when a pointer rests over any user (Users A-G) that shows real time health metrics of that user. Application 158 can be utilized to minimize the “fog of war” and general data uncertainties regarding a deployed situation requiring quick decisions. For example, in a forest fire-fighting situation, disaster relief (flood, hurricane, etc.) situation, and other such situations equipping agents with health monitoring devices 104 can help protect the safety of agents (Users A-G).

It should be appreciated, that monitoring client 156 is just one contemplated use of the health metric data. The health response system 140 is capable of receiving real-time (or delayed) health metrics form a monitored set of people deployed in any environment. A set of thresholds based upon heath metrics can be established and used to trigger any programmatically definable event. For example, the event can cause the health response system 140 to convey a digital message to a communication server (e.g., an email server, a telephony server, an IM server, etc.) that sends a message in response to alert a designated party that the threshold(s) have been exceeded.

Automated actions can also be taken relating to the environment including monitored individuals. For example, should health metrics indicate a high likelihood of a bank robbery attempt (assuming cashiers wear health monitoring devices 104 that indicate these cashiers are all in a panic state) then bank vaults, cash repositories, and the like can be automatically locked by the response system 140 to prevent a robbery attempt from succeeding (knowledge of such an auto-locking system can also deter potential robbery attempts due to a decreased likelihood of success). In a telemarketing or customer service example, a phone agent (wearing device 104) who becomes agitated (based upon monitored metrics) can have a current communication automatically transferred away from them for handling by a less agitated agent.

Basically, a combination of device 104 and system 140 provides a highly flexible and configurable capability for situation handling. It can be adapted for any situation where action(s) are to be taken based upon changes in health metrics. Advantageously, these actions can occur in a user 102 transparent fashion without requiring an active action on the part of the user 102.

Local hub 152 can be a local device in which can locally connect to health monitoring devices 104. For example, each of the health monitoring devices 155 can use a relatively short range wireless area network sufficient to communicate with the hub 152. For example, BLUETOOTH, WIFI, WIRELESS USB, ZIGBEE, and other standardized communication protocols can be used to connect the devices 104 to a hub 152, which is capable of communicating with network 150. In one contemplated embodiment, each health monitoring device 104 can function as a repeater for signals from other proximate devices 104, so that so long as an ad-hoc network is interconnected to the hub 152, communications are possible between the system 154 and each device 104 participating within the ad-hoc network.

Network 150 can include any hardware/software/and firmware necessary to convey digital content encoded within carrier waves. Content can be contained within analog or digital signals and conveyed through data or voice channels and can be conveyed over a personal area network (PAN) or a wide area network (WAN). The network 150 can include local components and data pathways necessary for communications to be exchanged among computing device components and between integrated device components and peripheral devices. The network 150 can also include network equipment, such as routers, data lines, hubs, and intermediary servers which together form a packet-based network, such as the Internet or an intranet. The network 150 can further include circuit-based communication components and mobile communication components, such as telephony switches, modems, cellular communication towers, and the like. The network 150 can include line based and/or wireless communication pathways.

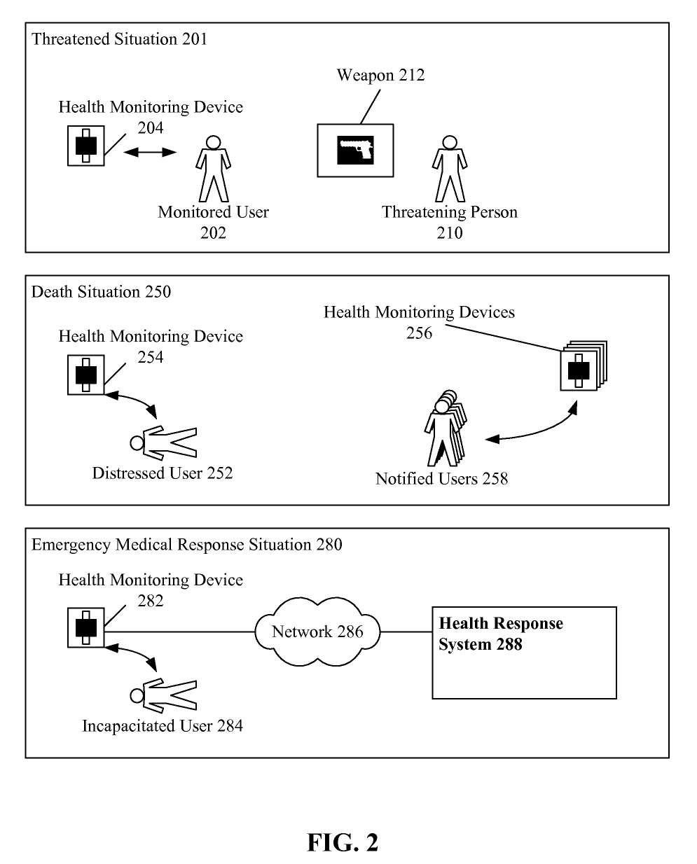

FIG. 2 illustrates scenarios 201, 250, 280 for remotely monitoring changes in bodily functions and responding to emergency situations in accordance with an embodiment of the inventive arrangements disclosed herein. In FIG. 2, health monitoring devices 204, 254, and 282 can be in context of health monitoring device 104 of system 100. The illustrated situations 201, 250, 280 can include threatened situation 201, death situation 250, and emergency medical response situation 280. Other situations are contemplated and the invention is not to be construed as limited to the sample situations 201, 250, 280. In all of the situations 201, 250, 280 health monitoring can be performed in real-time, near real-time, and/or after an appreciable delay depending upon implementation specifics.

Threatened situation 201 can include monitored user 202 and threatening person 210. Health monitoring device 204 can be monitoring health metrics of monitored user 202. Threatening person 210 can be using weapon 212 in a threatening manner towards monitored user 202. In situation 201, health monitoring device 204 can detect a change in monitored user 202's health metrics because user 202 is being threatened. The weapon 212 itself is extraneous (and not explicitly detected in situation 201) but is instead used as an example of an environmental factor likely to increase a heart rate (or other heath metric) of the monitored user 202.

The change in health metrics can cause an alert and health monitoring device 204 can contact other proximate health monitoring devices and/or a health response system (i.e. a system in context of health response system 140 of system 100). When multiple monitored users 202 are proximately located, heath metrics of the other users can be used to decrease a likelihood of false positive reactions. For example, a likelihood of a threatening situation 201 can be statistically high when all monitored users 202 in a geographic region exhibit consistent changed health metrics characteristic of fear (i.e., when all tellers of a bank exhibit increased heart rate past a designated threshold, a likelihood that a bank is experiencing a robbery or other emergency can be relatively high.)

Death situation 250 can include distressed user 252, who is being monitored by health monitoring device 254. Distressed user 252 can be dead (no pulse) in situation 250. Distressed user 252 can be related to notified users 258. For example, users 252 and 258 can be part of a military unit or bodyguards. In situation 250, health monitoring device 254 can detect that distressed user 252 has passed away and can notify proximate health monitoring devices 256. When health monitoring devices 256 receive notification of user 252's death, notified users 258 can be alerted of the change and they can respond accordingly.

Another reasonable use of health monitoring involving a death situation 250 can be in an emergency response context. For example, firefighters can be provided health metrics of users 252 trapped in a burning building. The health metrics can not only permit firefighters to pinpoint locations of users in distress, but can inform the firefighters when threatened individuals expire, so that firefighters will not risk their lives trying to save others who are beyond help.

Emergency medical response situation 280 can illustrate incapacitated user 284 being monitored by health monitoring device 282. User 284 can have been subject to an accident or health defect in which has left user 284 incapacitated. For example, user 284 can have fallen down stairs, experienced a heart attack or stroke, fainted or had a seizure, or the like. Health monitoring device 282 can detect the negative change in user 284's health and can communicate the change to health response system 288 via network 286. Health response system 288 can automatically dispatch an emergency medical response unit to aid user 284. Health response system 288 can be in context of health response system 140 of system 100. Network 286 can be in context of network 150 of system 100.

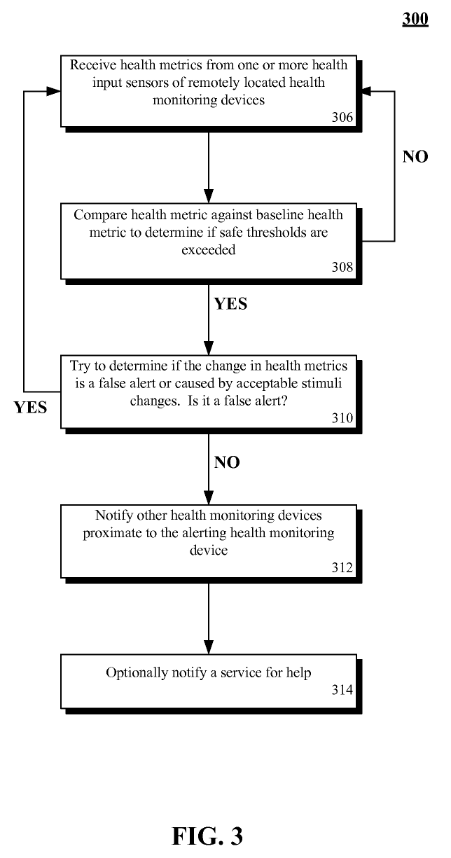

FIG. 3 is a flow chart of a method 300 for remotely monitoring changes in bodily functions and responding to emergency situations in accordance with an embodiment of the inventive arrangements disclosed herein.

Method 300 can be performed in context with system 100 and can begin in step 306, where health metrics can be received from one or more health input sensors of remotely located health monitoring devices. These health metrics can be received by a health monitoring system. In step 308, health metrics can be compared against baseline health metrics to determine if safe thresholds are exceeded. These thresholds can be based upon configurable values. If in step 308, safe thresholds have not been exceeded, method 300 can loop back to step 306, where health metrics can be received from remotely located health monitoring devices again.

If in step 308, safe thresholds have been exceeded, method 300 can continue to step 310, where the health metrics can be used to attempt to automatically detect a false alert. In some cases, a false alert can be caused by device malfunction or interference. In other cases, a false alert can be caused by temporary changes in bodily functions, such as a cough or sneeze, or an emotional response to things such as a joke, misstep, or the like. It is contemplated that there can be user configurable settings to prevent false alerts. For example, a user can be able to specify an acceptable range of fluctuation for certain bodily functions (i.e. heart rate, blood pressure). In another example, a delay can be established before alerting, so in the case of an alert, the health metrics can be re-read and compared to safe thresholds. Further, health metrics from proximate individuals, who are also monitored, can be used to minimize false positives.

If in step 310, it is determined to be a false alert, method 300 can loop back to step 306, where health metrics can be received from remotely located health monitoring devices again. If in step 310, the alert is determined to be genuine, method 300 can continue to step 312, where other health monitoring devices proximate to the alerting health monitoring device can be notified of the alert. In some embodiments, the alerting health monitoring device can alert proximate devices directly. In other embodiments, the alert can be managed by a health monitoring system, which can handle alerting proximate health monitoring devices. Further, a remote server that receives heath metrics can be configured to take a programmatic action (also configurable) when a received health metric exceeds a programmatically defined threshold. These programmatic actions can notify a designated user or set of users, can trigger a situation mitigating action, can result in metrics being recorded, can notify communicatively linked devices of a problem, etc.

Method 300 can complete in step 314, where a service can optionally be notified for help (assuming the help notification is the configured programmatic action that is taken when the threshold is exceeded). For example, if the alert requires immediate medical attention, an emergency medical service can be contacted.

The diagrams in FIGS. 1-3 illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A method for utilizing health metrics comprising:
receiving health metrics from at least one person wearing a health monitoring device in an unencumbered fashion that permits free motion, wherein the health monitoring device wirelessly conveys the heath metrics to a remotely located computing device;
comparing the received health metrics against at least one configurable, yet previously established threshold;
inferring a change in a situation proximate to the at least one person based upon comparison results;
firing a programmatic event based upon the inferred change; and
initiating at least one programmatic action responsive to the firing of the programmatic event, wherein the programmatic action initiates a response to the inferred change.

2. The method of claim 1, wherein the receiving and comparing of metrics, the inferring the change, firing the programmatic event, and the initiating at least one programmatic action occur in real-time.

3. The method of claim 1, wherein the at least one person wearing the health monitoring device comprises a plurality of people, said method further comprising:
comparing the received health metrics of the plurality of people against the threshold to generate the comparison results from which the change is inferred.

4. The method of claim 1, wherein the inferred change indicates that a wearing of the health monitoring device is in need of help, wherein said programmatic action automatically conveys a notification message to a responder that indicates the need of the wearer, wherein the conveyance of the notification message occurs without the wearing taking an active action.

5. The method of claim 1, wherein the inferred change indicates a change in a user state, wherein user states able to be inferred comprise unconsciousness, death, panic, and normality.

6. The method of claim 1, wherein a plurality of different people wear the health monitoring device concurrently participate in a common endeavor, said programmatic action comprising a notification action, said method further comprising:
executing the notification action to automatically notify each of the different people of the inferred change, wherein the different people are notified if the inferred change in at least one of real-time and near real time.

7. The method of claim 1, a plurality of agents each wearing the health monitoring device, wherein the inferred change in the situation indicates a potential breach of a security perimeter established by said agents.

8. The method of claim 1, a plurality of agents each wearing the health monitoring device when deployed to a potentially hostile situation, wherein the inferred change is configured to immediate provide notification to at least one designated person when any of the plurality of agents is rendered unconscious and is configured to immediately provide a notification to at least one designated person when any of the plurality of agents becomes dead, wherein the provided notification occurs in at least one of real-time and near real-time.

9. The method of claim 1, wherein the wearing of the health monitoring device occurs in an employment setting, wherein a set of at least one employees are required to wear the health monitoring device during the course their employment, wherein the inferred change is configured to immediately provide notification to at least one designated person when an employee wearing the device enters a state of panic.

10. A computer program product for utilizing health metrics comprising:
a computer usable medium having computer usable program code embodied therewith, the computer usable program code comprising:
computer usable program code configured to receive health metrics from at least one person wearing a health monitoring device in an unencumbered fashion that permits free motion, wherein the health monitoring device wirelessly conveys the heath metrics to a remotely located computing device;
computer usable program code configured to compare the received health metrics against at least one configurable, yet previously established threshold;
computer usable program code configured to infer a change in a situation proximate to the at least one person based upon comparison results;
computer usable program code configured to fire a programmatic event based upon the inferred change; and
computer usable program code configured to initiate at least one programmatic action responsive to the firing of the programmatic event, wherein the programmatic action initiates a response to the inferred change.

11. The computer program product of claim 10, wherein the receiving and comparing of metrics, the inferring the change, firing the programmatic event, and the initiating at least one programmatic action occur in real-time.

12. The computer program product of claim 10, wherein the at least one person wearing the health monitoring device comprises a plurality of people; said computer program product further comprising:
computer usable program code configured to compare the received health metrics of the plurality of people against the threshold to generate the comparison results from which the change is inferred.

13. The computer program product of claim 10, wherein the inferred change indicates that a wearing of the health monitoring device is in need of help, wherein said programmatic action automatically conveys a notification message to a responder that indicates the need of the wearer, wherein the conveyance of the notification message occurs without the wearing taking an active action.

14. The computer program product of claim 10, wherein the inferred change indicates a change in a user state, wherein user states able to be inferred comprise unconsciousness, death, panic, and normality.

15. The computer program product of claim 10, wherein a plurality of different people wear the health monitoring device concurrently participate in a common endeavor, said programmatic action comprising a notification action, said computer program product further comprising:
computer usable program code configured to execute the notification action to automatically notify each of the different people of the inferred change, wherein the different people are notified if the inferred change in at least one of real-time and near real time.

16. The computer program product of claim 10, a plurality of agents each wearing the health monitoring device, wherein the inferred change in the situation indicates a potential breach of a security perimeter established by said agents.

17. The computer program product of claim 10, a plurality of agents each wearing the health monitoring device when deployed to a potentially hostile situation, wherein the inferred change is configured to immediate provide notification to at least one designated person when any of the plurality of agents is rendered unconscious and is configured to immediately provide a notification to at least one designated person when any of the plurality of agents becomes dead, wherein the provided notification occurs in at least one of real-time and near real-time.

18. The computer program product of claim 10, wherein the wearing of the health monitoring device occurs in an employment setting, wherein a set of at least one employees are required to wear the health monitoring device during the course their employment, wherein the inferred change is configured to immediately provide notification to at least one designated person when an employee wearing the device enters a state of panic.
